(19) United States
(12) Patent Application Publication
(10) Pub. No.: US 2023/0093363 A1
(43) Pub. Date: Mar. 23, 2023

(54) LIGHT-EMITTING PARTICLES

(71) Applicant: Sumitomo Chemical Co., Ltd., Tokyo (JP)

(72) Inventor: Jonathan Behrendt, Godmanchester (GB)

(73) Assignee: Sumitomo Chemical Co., Ltd., Tokyo (JP)

(21) Appl. No.: 17/909,251

(22) PCT Filed: Mar. 2, 2021
(86) PCT No.: PCT/GB2021/050522

§ 371 Date: Sep. 2, 2022

(30) Foreign Application Priority Data
Mar. 3, 2020 (GB) 2003068.0

(51) Int. Cl. C09K 11/06; C09K 11/02; G01N 33/58

(52) U.S. Cl. CPC C09K 11/06; C09K 11/025; G01N 33/582; B82Y 15/00

(57) ABSTRACT

A light-emitting particle comprising a core and a composite shell layer in contact with and surrounding the core wherein the composite shell layer comprises silica and a light-emitting polymer distributed across the thickness of the composite shell layer.

### BACKGROUND

Embodiments of the present disclosure relate to light-emitting particles; methods of forming the same; and the use thereof as a luminescent marker.

### BACKGROUND

Light-emitting polymers have been disclosed as labelling or detection reagents.

Geng et al, “A general approach to prepare conjugated polymer dot embedded silica nanoparticles with a SiO2@CP@SiO2 structure for targeted HER2-positive cellular imaging”, Nanoscale, 2013, vol. 5, pp 8593-8601, describes silica-conjugated polymer (CP) nanoparticles having a “SiO₂@CP@SiO₂” structure.

Shenoi-Perdoor et al, “Red-emitting fluorescent organic@silicate core-shell nanoparticles for bio-imaging”, New Journal of Chemistry, 2018, 42, 15353-15360 discloses nanoparticles with a fluorescent organic core surrounded by a silicate shell.

EP 2626960 discloses an active laser medium of a metal nanoparticle and shell including a luminophor. A luminescence spectrum of the luminophor overlaps with a peak surface plasmon resonance of the metal nanoparticle.

Y Chan et al, “Incorporation of Luminescent Nanocrystals into Monodisperse Core-Shell Silica Microspheres”, Adv. Mater. Volume 16, Issue 23-24, December 2004 pages 2092-2097 discloses silica microspheres coated with a silica or titania shell containing fluorescent semiconductor nanocrystals.

Behrendt et al, PLoS One 2013, 8 (3), e50713 discloses core-shell polymer microspheres for use in in vitro bioimaging and biomolecule delivery.

### SUMMARY

In some embodiments, there is provided a light-emitting particle comprising a core and a composite shell layer in contact with and surrounding the core. The composite shell layer comprises or consists of silica and a light-emitting polymer distributed across the thickness of the composite shell layer.

Optionally, the core has a single core layer.

Optionally, the core comprises silica.

Optionally, the light-emitting particle is a nanoparticle having a diameter of less than 1000 nm.

Optionally, the core and the composite shell layer form a nucleus of the light-emitting particle and wherein the composite shell layer is the outermost layer of the light-emitting particle nucleus.

Optionally, at least one surface group is bound to the composite shell layer.

Optionally, a biomolecule binding group bound to the composite shell layer.

In some embodiments, there is provided a powder comprising or consisting of light-emitting particles as described herein.

In some embodiments, there is provided a dispersion comprising or consisting of light-emitting particles as described herein dispersed in a liquid.

In some embodiments, there is provided a method of forming a light-emitting particle having a core and a composite shell layer in contact with and surrounding the core wherein the composite shell layer comprises or consists of a light-emitting polymer and silica. According to this method, the composite shell layer is formed by polymerising a monomer for forming the silica in the presence of the core and the light-emitting polymer in dissolved form.

In some embodiments, there is provided a method of marking a biomolecule comprising the step of binding the biomolecule to a light-emitting marker particle as described herein.

In some embodiments, there is provided an assay method for a target analyte in which a sample is contacted with light-emitting marker particles described herein, and determining any binding of the target analyte to the light-emitting marker.

Optionally, the sample contacted with the light-emitting marker particles is analysed by flow cytometry. Optionally, an amount of target analyte bound to the light-emitting marker particles is determined. Optionally, the sample comprises a mixture of cells and one or more different types of target cells bound to the light-emitting marker are identified and/or quantified.

Optionally, the method of marking a biomolecule includes separating target analyte bound to the light-emitting particles from the target analyte which is not bound to the light-emitting particles.

In some embodiments, the present disclosure provides a method of sequencing nucleic acids comprising:

contacting a primed template nucleic acid molecule with a polymerase and a test nucleotide; incorporating the test nucleotide into a primed strand of the primed template only if it comprises a base complementary to the next base of the template strand;

irradiating the primed strand; and

determining from luminance of the primed strand if the test nucleotide has been incorporated into the primed strand,

wherein the test nucleotide of the irradiated primed strand is bound to a light-emitting particle as described herein.

In some embodiments, the test nucleotide contacted with the polymerase and nucleic acid molecule is bound to the light-emitting particle.

In some embodiments, the light-emitting marker binds to the test nucleotide after incorporation of the test nucleotide into the primed strand.

### DESCRIPTION OF THE DRAWINGS

The disclosed technology and accompanying figures describe some implementations of the disclosed technology.

FIG. 1 is a schematic illustration of a particle according to some embodiments in which the particle has a single layer core and a composite shell;

FIG. 2 is a schematic illustration of a method according to some embodiments for forming the particle of FIG. 1;

FIG. 3 is a schematic illustration of a particle according to some embodiments in which the particle has two core layers and a composite shell;

FIG. 4 is a graph of size distribution of a 60 nm silica nanoparticle before and after formation of a composite shell according to some embodiments of the present disclosure;

FIG. 5 is a graph of size distribution of a 120 nm silica nanoparticle before and after formation of a composite shell according to some embodiments of the present disclosure;

FIG. 6 is a graph of size distribution of a comparative 120 nm silica nanoparticle before and after adsorption of a light-emitting polymer and formation of a silica shell;

FIG. 7 shows UV-visible absorption spectra for particles of FIGS. 4-6; and

FIG. 8 shows photoluminescent spectra for particles of FIGS. 4-6.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, electromagnetic, or a combination thereof. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to an atom include any isotope of that atom unless stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while some aspect of the technology may be recited as a computer-readable medium claim, other aspects may likewise be embodied as a computer-readable medium claim, or in other forms, such as being embodied in a means-plus-function claim.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

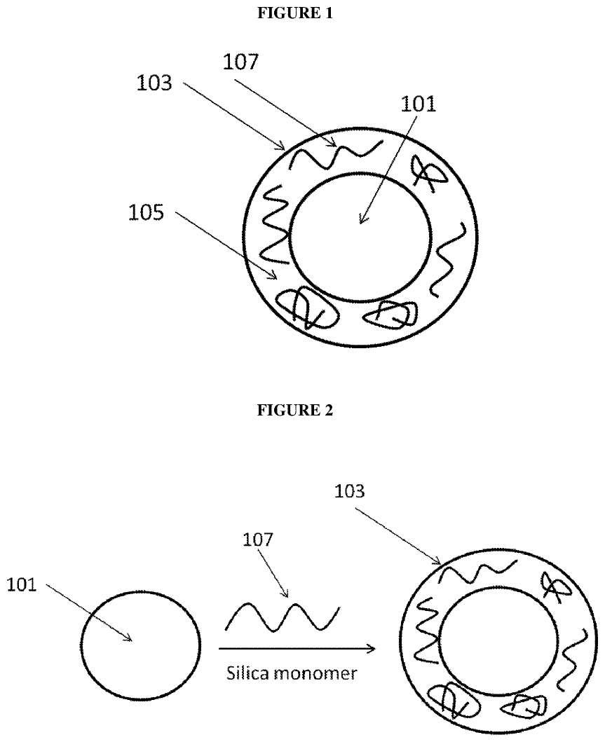

FIG. 1 schematically illustrates a light-emitting particle according to some embodiments of the present disclosure.

The light-emitting particle has a core 101 and a composite shell layer 103 containing silica 105 and at least one light-emitting polymer 107 distributed therein.

The core may consist of a single material or may contain a mixture of two or more materials.

The material or materials of the core may be selected from organic and inorganic materials.

Inorganic core materials include, without limitation one or more of:
Silica
Elemental metals, e.g. gold or iron;
Metal oxides, e.g. iron oxide or alumina; and
Quantum dot nanoparticles, e.g. CdSe or PbS.

Organic core materials may be selected from electrically insulating polymers, e.g. polystyrene and polyacrylates, e.g. poly(methylmethacrylate).

In some embodiments, the core has a property allowing manipulation of the particle, e.g. separation of the particles from a mixture containing the particles, either alone or in combination with a functionality provided by the composite shell. In some embodiments, the core comprises a magnetic material, e.g. metallic iron or an iron compound.

Optionally, the core comprises or consists of silica.

The light-emitting particle has a composite shell layer 103 containing at least one light-emitting polymer and silica. Optionally, the composite shell layer contains only one light-emitting polymer. Optionally, the composite shell layer consists of the at least one light-emitting polymer and silica. Optionally, the composite shell further comprises one or more non-polymeric light-emitting materials.

The silica:organic light-emitting polymer weight ratio of the composite shell may be in the range of about 1:3-1:300.

The organic light-emitting polymer may be uniformly distributed throughout the thickness of the composite shell layer.

In some embodiments, some of the light-emitting polymer in the composite shell layer is covalently or non-covalently bound to the core, the remaining light-emitting polymer in the composite shell not being bound to the core. The remaining light-emitting polymer in the composite shell is optionally not in direct contact with the core.

In some embodiments, none of the light-emitting polymer chains are bound to the core.

The individual light-emitting polymer chains of the light-emitting polymer may each independently have any configuration within the composite shell including, without limitation, a folded or unfolded configuration. The configuration of the light-emitting polymer chains may be affected by the composite shell formation process and conditions.

The present inventors have found that a composite shell layer as described herein can be formed to a wide and controllable range of thicknesses onto a wide range of cores. Particles containing such a composite shell layer may have a higher brightness than particles in which all light-emitting polymer is bound to the core.

In some embodiments, there is no light-emitting material present in the core.

In some embodiments, the core contains a light-emitting material which is different from the light-emitting polymer(s) of the composite shell layer.

Optionally, the core modulates the optical properties of the light-emitting polymer contained in the shell.

In some embodiments, the core comprise a metal configured to undergo surface plasmon resonance stimulated by light emitted from the one or more light-emitting polymers disposed in the composite shell.

In some embodiments, the core does not comprise a material configured to undergo surface plasmon resonance stimulated by light emitted from the one or more light-emitting polymers disposed in the composite shell. Optionally, the core does not comprise any electrically conducting materials. Optionally, the core consists of one or more electrically insulating materials.

In some embodiments, the core comprises a material configured to transfer energy to the light-emitting polymer contained in the shell, e.g. by Forster resonance energy transfer such as from a quantum dot disposed in the core to an organic light-emitting polymer disposed in the composite shell.

Optionally, the particles are nanoparticles.

Preferably, the particles have a number average diameter of no more than 60 microns.

Preferably, the particles have a number average diameter of at least 10 nm.

Preferably, the cores have a number average diameter of no more than 50 microns.

Preferably, the cores have a number average diameter of at least 2 nm.

Preferably, the composite shells have an average thickness of no more than 1 micron.

Preferably, the composite shells have a thickness of at least 5 nm.

Number average diameters provided herein are as measured by a Malvern Zetasizer Nano ZS.

Average composite shell thickness as provided herein is given by (number average particle diameter−number average core diameter)/2.

Particles as described herein may be provided as a powder. In some embodiments, the particles may be stored in a dry, optionally lyophilised, form. The particles may be stored in a frozen form.

Particles as described herein may be provided in a colloidal suspension comprising the particles suspended in a liquid. Preferably, the liquid is selected from water, C₁₋₈ alcohols and mixtures thereof. Preferably, the particles form a uniform (non-aggregated) colloid in the liquid. The liquid may be a solution comprising salts dissolved therein, optionally a buffer solution.

FIG. 2 illustrates a process for forming a particle according to some embodiments of the present disclosure. A monomer for forming silica, e.g. an alkoxysilane, is polymerised in a liquid containing the monomer, the particle core and the light-emitting polymer. The light-emitting polymer may be dissolved in the liquid, which preferably comprises or consists of one or more protic solvents. Upon polymerisation of the monomer, the silica forms as a shell around the core, with the light-emitting polymer being incorporated into the shell.

The thickness of the composite shell may be controlled by one or both of concentration of silica monomer and polymerisation time.

The silica:light-emitting polymer weight ratio of the composite shell may be controlled by selection of the silica monomer:light-emitting polymer weight ratio. The silica:light-emitting polymer weight ratio may be determined by determining the weights of unreacted monomer and unreacted light-emitting polymer following shell formation and subtracting these weights from the corresponding starting weights.

Optionally, light-emitting particles as described herein emit light in the visible range of the electromagnetic spectrum when excited by an energy source, e.g. a light source.

Emission from the light-emitting particles may have a peak wavelength in the range of 350-1,000 nm. Emission in the visible range may comprise or consist of red, green or blue light or a mixture thereof.

A blue light-emitting particle may have a photoluminescence spectrum with a peak of no more than 500 nm, preferably in the range of 400-500 nm, optionally 400-490 nm.

A green light-emitting particle may have a photoluminescence spectrum with a peak of more than 500 nm up to 580 nm, optionally more than 500 nm up to 540 nm.

A red light-emitting particle may have a photoluminescence spectrum with a peak of no more than more than 580 nm up to 630 nm, optionally 585 nm up to 625 nm.

The photoluminescence spectrum of light-emitting particles as described herein may be as measured using an Ocean Optics 2000+ spectrometer.

Optionally, light-emission from the light-emitting particles is observed upon irradiation with a light source having a peak wavelength in the range of 220-1800 nm. UV/vis absorption spectra of light-emitting particles as described herein may be as measured using a Cary 5000 UV-vis-IR spectrometer.

The light-emitting polymer may have a Stokes shift in the range of 10-850 nm.

FIG. 1 illustrates a particle having a single core layer. In other embodiments, the light-emitting particle has a core containing two or more core layers. It will be understood that a “core” as described herein is the or each core layer up to and including the core layer adjacent to the composite shell layer.

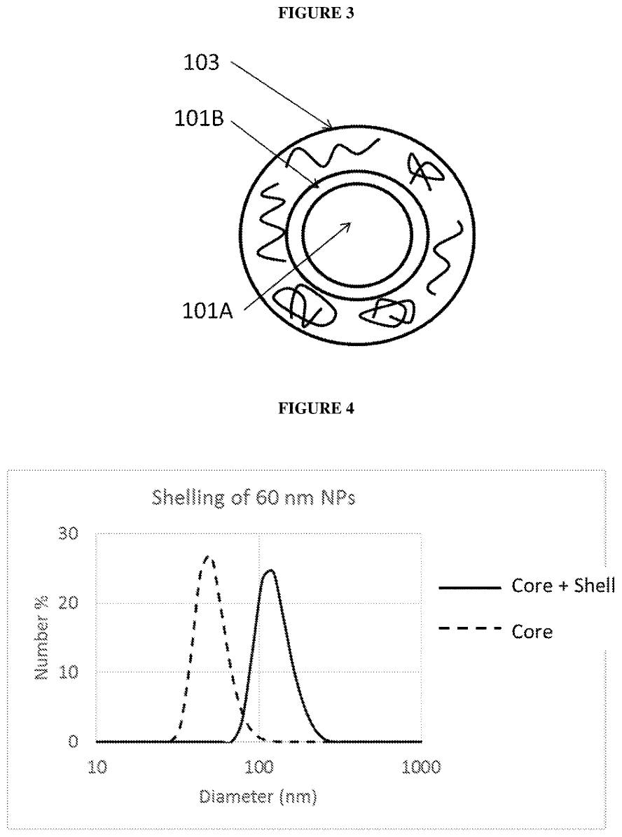

FIG. 3 illustrates a particle having a first core layer 101A and a second core layer 101B. The core layer adjacent to the composite shell layer, i.e. the second core layer 101B in FIG. 3, may be selected according to its compatibility with one or more materials of the composite shell layer, e.g. compatibility with silica. In some embodiments, the core layer adjacent to the composite shell layer comprises or consists of an amphiphilic polymer, e.g. poly(vinylpyrollidone).

FIGS. 1 and 2 illustrate particles in which composite shell layer is the outer layer. In other embodiments, one or more further layers are formed over the composite shell layer, e.g. a silica layer.

FIGS. 1 and 2 illustrate particles in which composite shell layer is the outer layer.

Light-Emitting Polymer

The light-emitting polymer may be fluorescent or phosphorescent.

The light-emitting polymer may have a solubility in water or a C₁₋₈ alcohol at 20° C. of at least 0.01 mg/ml, optionally at least 0.1, 1, 5 or 10 mg/ml. Optionally, solubility is in the range of 0.01-10 mg/ml.

The light-emitting polymer may have a solubility in a C₁₋₄ alcohol, preferably methanol, at 20° C. of at least 0.01 mg/ml, optionally at least 0.1, 1, 5 or 10 mg/ml.

Solubility may be as determined by visual observation under white and/or UV light after heating of a mixture of the solvent and the light-emitting polymer on a hotplate at 60° C. for 30 minutes with stirring and allowing the solution to cool to 20° C.

The polystyrene-equivalent number-average molecular weight (Mn) measured by gel permeation chromatography of light-emitting polymers described herein may be in the range of about 1×10³ to 1×10⁸, and preferably 1×10⁴ to 5×10⁶. The polystyrene-equivalent weight-average molecular weight (Mw) of the light-emitting polymers described herein may be 1×10³ to 1×10⁸, and preferably 1×10⁴ to 1×10⁷.

A light-emitting polymer as described herein may be a conjugated or non-conjugated light-emitting polymer. By “conjugated light-emitting polymer” as used herein is meant that a backbone of the polymer contains aromatic, heteroaromatic or vinylene groups which are directly conjugated to aromatic, heteroaromatic or vinylene groups of adjacent repeat units. The backbone may be conjugated along its entire length. The backbone may contain a plurality of conjugated sections which are not conjugated to one another.

A conjugated light-emitting polymer as described herein may contain one or more of an arylene repeat unit; a heteroarylene repeat unit; and an arylamine repeat unit, each of which may be unsubstituted or substituted with one or more substituents.

Substituents may be selected from non-polar substituents, for example C₁₋₃₀ hydrocarbyl substituents; and polar substituents. Polar substituents may be ionic or non-ionic. A polar substituent may confer on the light-emitting polymer a solubility in a C₁₋₈ alcohol at 20° C. of at least 0.1 mg/ml, optionally at least 0.2, 03 or 0.5 mg/ml.

Non-polar substituents include, without limitation, C₁₋₃₀ hydrocarbyl substituents, e.g. C₁₋₂₀ alkyl, unsubstituted phenyl and phenyl substituted with one or more C₁₋₂₀ alkyl groups.

An exemplary non-ionic polar groups has formula —O(R³O)_{q}—R⁴ wherein R³ in each occurrence is a C₁₋₁₀ alkylene group, optionally a C₁₋₅ alkylene group, wherein one or more non-adjacent, non-terminal C atoms of the alkylene group may be replaced with O, R⁴ is H or C₁₋₅ alkyl, and q is at least 1, optionally 1-10. Preferably, q is at least 2. More preferably, q is 2 to 5. The value of q may be the same in all the polar groups of formula —O(R³O)_{q}—R⁴. The value of q may differ between non-ionic polar groups of the same polymer.

By “C₁₋₅ alkylene group” as used herein with respect to R³ is meant a group of formula —(CH₂)_{f}— wherein f is from 1-5.

Optionally, the polymer comprises non-ionic polar groups of formula —O(CH₂CH₂O)_{q}R⁴ wherein q is at least 1, optionally 1-10 and R⁴ is a C₁₋₅ alkyl group, preferably methyl. Preferably, q is at least 2. More preferably, q is 2 to 5, most preferably q is 3.

Ionic substituents may be anionic or cationic.

Exemplary anionic groups are —COO⁻, a sulfonate group; hydroxide; sulfate; phosphate; phosphinate; or phosphonate. The counter cation of an anionic group may be selected from a metal cation, optionally Li⁺, Na⁺, K⁺, Cs⁺, preferably Cs⁺, and an organic cation, optionally ammonium, such as tetraalkylammonium, ethylmethyl imidazolium or pyridinium.

An exemplary cationic group is —N(R⁵)₃⁺ wherein R⁵ in each occurrence is H or C₁₋₁₂ hydrocarbyl. Preferably, each R⁵ is a C₁₋₁₂ hydrocarbyl, e.g. C₁₋₁₂ alkyl; phenyl; or phenyl substituted with one or more C₁₋₆ alkyl groups. The counter anion of a cationic group may be a halide; a sulfonate group, optionally mesylate or tosylate; hydroxide; carboxylate; sulfate; phosphate; phosphinate; phosphonate; or borate.

A polar substituent may have formula -Sp-(R¹)n wherein Sp is a spacer group; n is at least 1, optionally 1, 2, 3 or 4; and; R¹ in each occurrence is independently an ionic or non-ionic polar group.

Preferably, Sp is selected from:
C₁₋₂₀ alkylene or phenylene-C₁₋₂₀ alkylene wherein one or more non-adjacent C atoms may be replaced with O, S, N or C═O;
a C₆₋₂₀ arylene or 5-20 membered heteroarylene, more preferably phenylene, which, other than the one or more substituents R¹, may be unsubstituted or substituted with one or more non-polar substituents, optionally one or more C₁₋₂₀ alkyl groups.

“alkylene” as used herein means a branched or linear divalent alkyl chain.

Optionally, the conjugated light-emitting polymer contains one or more arylene repeat units selected from C₆₋₂₀ arylene repeat units, e.g. phenylene fluorene, indenofluorene, benzofluorene, dihydrophenanthrene, phenanthrene, naphthalene and anthracene repeat units.

Optionally, the polymer contains one or more arylene repeat units selected from formulae (III)-(VI):

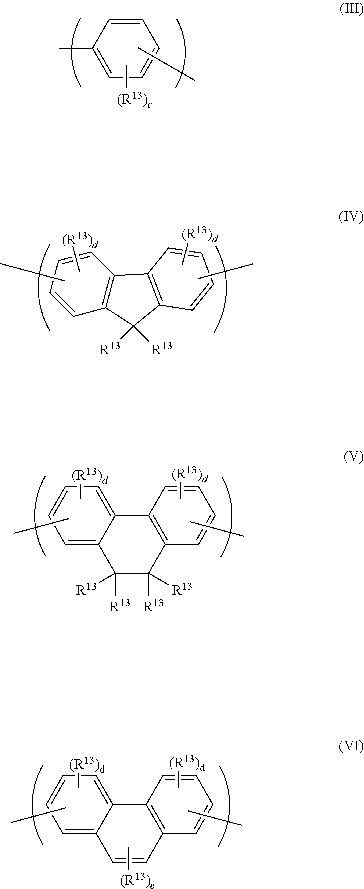

wherein R¹³ in each occurrence is independently a substituent and two R¹³ groups may be linked to form a ring; c is 0, 1, 2, 3 or 4, preferably 1 or 2; each d is independently 0, 1, 2 or 3, preferably 0 or 1; and e is 0, 1 or 2, preferably 2.

Each R¹³ group, where present, may be selected from a non-polar or polar substituent as described herein.

In some embodiments, two R¹³ groups may be linked to form a 6-membered ring or 7-membered ring. Optionally, two R¹³ groups are linked to form a ring in which the linked R¹³ groups form a C₄- or C₅-alkylene chain wherein one or more non-adjacent C atoms of the alkylene chain may be replaced with O, S, NR¹⁰ or Si(R¹⁰)₂ wherein R¹⁰ in each occurrence is independently a C₁₋₂₀ hydrocarbyl group.

An exemplary repeat unit in which two R¹³ groups are linked has formula (IVb):

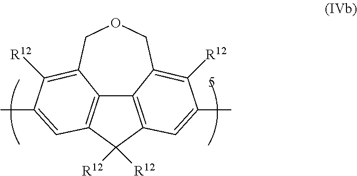

wherein each R¹² is independently H or R¹³, preferably H.

In some embodiments, no R groups are linked to one another.

A preferred arylene repeat unit has formula (IVa):

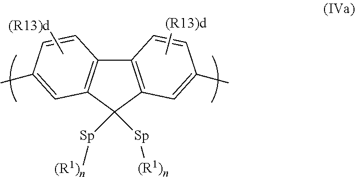

An exemplary repeat unit of formula (IVa) is:

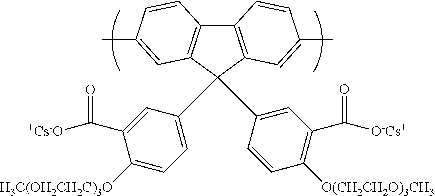

Repeat units comprising or consisting of one or more unsubstituted or substituted 5-20 membered heteroarylene groups in the polymer backbone include, without limitation, thiophene repeat units, bithiophene repeat units, benzothiadiazole repeat units, and combinations thereof. Exemplary heteroarylene repeat units include repeat units of formulae (VIII)-(XI):

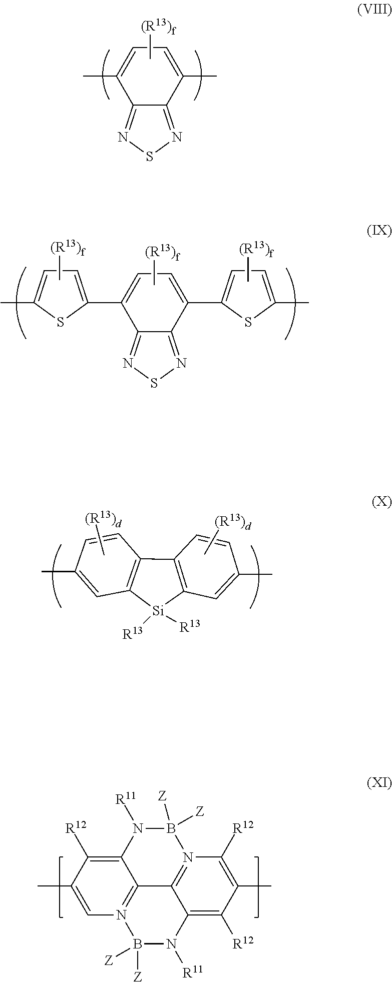

wherein R¹¹ independently in each occurrence is a C₁₋₂₀ hydrocarbyl group; Z in each occurrence is independently a substituent, preferably F or a C₁₋₂₀ hydrocarbyl group; and R¹², R¹³ and d are as described above.

A C₁₋₂₀ hydrocarbyl group as described anywhere herein is optionally selected from C₁₋₂₀ alkyl, unsubstituted phenyl and phenyl substituted with one or more C₁₋₁₂ alkyl groups.

wherein R¹³ in each occurrence is independently a substituent and f is 0, 1 or 2. Each R¹³ may independently be selected from polar and non-polar substituents as described above.

Arylamine repeat units may have formula (XII):

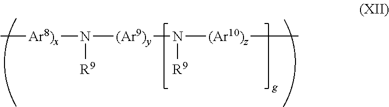

wherein Ar⁸, Ar⁹ and Ar¹⁰ in each occurrence are independently selected from substituted or unsubstituted aryl or heteroaryl, g is 0, 1 or 2, preferably 0 or 1, R¹³ independently in each occurrence is a substituent, and x, y and z are each independently 1, 2 or 3.

R⁹, which may be the same or different in each occurrence when g is 1 or 2, is preferably selected from the group consisting of alkyl, optionally C₁₋₂₀ alkyl, Ar¹¹ and a branched or linear chain of Ar¹¹ groups wherein Ar¹¹ in each occurrence is independently substituted or unsubstituted aryl or heteroaryl.

Any two aromatic or heteroaromatic groups selected from Ar⁸, Ar⁹, and, if present, Ar¹⁰ and Ar¹¹ that are directly bound to the same N atom may be linked by a direct bond or a divalent linking atom or group. Preferred divalent linking atoms and groups include O, S; substituted N; and substituted C.

Ar⁸ and Ar¹⁰ are preferably C₆₋₂₀ aryl, more preferably phenyl, which may be unsubstituted or substituted with one or more substituents.

In the case where g=0, Ar⁹ is preferably C₆₋₂₀ aryl, more preferably phenyl, that may be unsubstituted or substituted with one or more substituents.

In the case where g=1, Ar⁹ is preferably C₆₋₂₀ aryl, more preferably phenyl or a polycyclic aromatic group, for example naphthalene, perylene, anthracene or fluorene, which may be unsubstituted or substituted with one or more substituents. It is particularly preferred that Ar⁹ is anthracene when g=1.

R⁹ is preferably Ar¹¹ or a branched or linear chain of Ar¹¹ groups. Ar¹¹ in each occurrence is preferably phenyl that may be unsubstituted or substituted with one or more substituents.

Exemplary groups R⁹ include the following, each of which may be unsubstituted or substituted with one or more substituents, and wherein * represents a point of attachment to N:

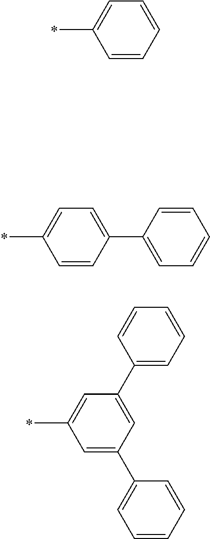

x, y and z are preferably each 1.

Ar⁸, Ar⁹, and, if present, Ar¹⁰ and Ar¹¹ are each independently unsubstituted or substituted with one or more, optionally 1, 2, 3 or 4, substituents.

Substituents may independently be selected from non-polar or polar substituents as described herein.

Preferred substituents of Ar⁸, Ar⁹, and, if present, Ar¹⁰ and Ar¹¹ are C₁₋₄₀ hydrocarbyl, preferably C₁₋₂₀ alkyl.

Preferred repeat units of formula (XII) include unsubstituted or substituted units of formulae (XII-1), (XII-2) and (XII-3):

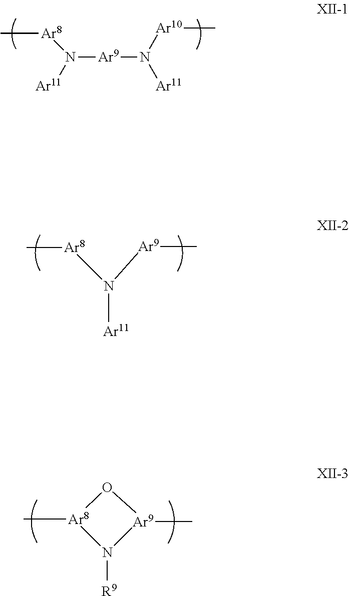

Conjugated light-emitting polymers as described herein may be formed by polymerising monomers comprising leaving groups that leave upon polymerisation of the monomers to form conjugated repeat units. Exemplary polymerization methods include, without limitation, Yamamoto polymerization as described in, for example, T. Yamamoto, “Electrically Conducting And Thermally Stable pi-Conjugated Poly(arylene)s Prepared by Organometallic Processes”, Progress in Polymer Science 1993, 17, 1153-1205, the contents of which are incorporated herein by reference and Suzuki polymerization as described in, for example, WO 00/53656, WO 2003/035796, and U.S. Pat. No. 5,777,070, the contents of which are incorporated herein by reference.

Composite Shell Formation

The composite shell may be formed by reacting a monomer for forming silica in the presence of the core and dissolved organic light-emitting polymer (s) to be incorporated into the composite shell.

Optionally, the silica monomer is an alkoxysilane, preferably a trialkoxy or tetra-alkoxysilane, optionally a C₁₋₁₂ trialkoxy or tetra-alkoxysilane, for example tetraethyl orthosilicate. The silica monomer may be substituted only with alkoxy groups or may be substituted with one or more groups.

Optionally, the silica monomer is polymerised in a liquid comprising or consisting of an ionic solvent or a protic solvent, preferably a solvent selected from water, alcohols and mixtures thereof. Exemplary alcohols include, without limitation, methanol, ethanol, 1-propanol, isopropanol, 1-butanol, 2-butanol, t-butanol and mixtures thereof. Preferably the solution comprises or consists of an alcoholic solvent selected from methanol, ethanol, isopropanol or mixtures thereof, more preferably the solution comprises or consists of a solvent selected from methanol, ethanol or mixtures thereof. Preferably, the solvent system does not comprise a non-alcoholic solvent other than water.

Polymerisation may be carried out in the presence of a base, e.g. a metal hydroxide, preferably alkali metal hydroxide, ammonium hydroxide or tetraalkylammonium hydroxide.

The particles may be isolated following formation of the composite shell and resuspended in an aqueous solvent, an organic solvent or a mixture thereof. The composite particles may be isolated from the reaction mixture by centrifuging.

Surface Groups

The core and the composite shell, along with any further shell layers, may form a nucleus of the light-emitting particle. One or more surface groups may be bound, e.g. covalently bound, to the outer surface of the nucleus.

In some embodiments, a biomolecule binding group is bound to an outer surface of the particle, which may be an outer surface of the composite shell or a further shell layer formed over the composite shell. The biomolecule binding group may be bound directly to the surface of the particle bound through a surface binding group.

The light-emitting particle may comprise two or more different surface groups. Optionally, one surface group comprises a biomolecule binding group and another surface group does not comprise the biomolecule binding group. The, or each, surface group may comprise a polyether chain. By “polyether chain” as used herein is meant a divalent chain comprising a plurality of ether groups, e.g. a polyethylene glycol chain.

The biomolecule binding group may be configured to bind to a target biomolecule, or to bind to a binding agent having an affinity for the biomolecule. Target biomolecules include without limitation DNA, RNA, peptides, carbohydrates, antibodies, antigens, enzymes, proteins and hormones. It will be understood that the biomolecule binding group may be selected according to the target biomolecule or binding agent.

A preferred biomolecule binding group is biotin. In some embodiments, the biotin biomolecule binding group binds directly to a target analyte.

In some embodiments, the biotin biomolecule binding group is bound to a protein having a plurality of biotin binding sites, preferably streptavidin, neutravidin, avidin or a recombinant variant or derivative thereof and biotinylated biomolecule having a second biotin group is bound to the same protein. The biotinylated biomolecule may be selected according to the target analyte. The biotinylated biomolecule may comprise an antigen binding fragment, e.g. an antibody, which may be selected according to a target antigen.

In some embodiments, the biomolecule binding group is formed on the surface of the particle after formation of the composite shell. In some embodiments, the silica monomer for forming silica of the composite shell is substituted with a reactive binding group which does not react during polymerisation of the silica monomer or which is protected during polymerisation of the silica monomer and which may be deprotected following formation of the composite shell.

Applications

Light-emitting particles as described herein may be used as luminescent probes in an immunoassay such as a lateral flow or solid state immunoassay. Optionally the light emitting particles are for use in fluorescence microscopy, flow cytometry, nucleic acid sequencing methods for example next generation sequencing, in-vivo imaging, or any other application where a light-emitting marker configured to bind to a target analyte is brought into contact with a sample to be analysed. The applications can medical, veterinary, agricultural or environmental applications whether involving patients (where applicable) or for research purposes.

In use as light-emitting marker particles, the light-emitting particles may be irradiated at an absorption wavelength of the light-emitting polymer or, if present, an absorption wavelength of a material configured to transfer energy to the light-emitting polymer, and emission from the light-emitting polymer may be detected.

In some embodiments, the sample following contact with the particles is analysed by flow cytometry. In flow cytometry, the particles are irradiated by at least one wavelength of light, optionally two or more different wavelengths, e.g. one or more wavelengths including at least one of 355, 405, 488, 530, 562 and 640 nm±10 nm. Light emitted by the particles may be collected by one or more detectors. Detectors may be selected from, without limitation, photomultiplier tubes and photodiodes. To provide a background signal for calculation of a staining index, measurement may be made of particles mixed with cells which do not bind to the particles.

In some embodiments, a target analyte may be immobilised on a surface carrying a group capable of binding to the target analyte, either before or after the target analyte binds to the particles. The particles bound to the target analyte immobilised on the surface may then be separated from any light-emitting particles which are not bound to the target analyte.

In the case where the light-emitting particles are used in a nucleic acid sequencing method, the surface of the light-emitting particle may carry a group capable of binding to a nucleotide to form a test nucleotide. For example, one of the test nucleotide and the light-emitting particle may be functionalised with biotin and the other of the test nucleotide and the conjugated polymer may be functionalised with avidin, streptavidin, neutravidin or a recombinant variant thereof.

### EXAMPLES

### Particle Example 1

60 nm Silica nanoparticles having a number average diameter of 60 nm (200 μL, 10 mg/mL in water, purchased from Nanocomposix), 200 μL deionised water, Light-Emitting Polymer 1 in methanol (500 μL, 1 mg/mL), methanol (434 μL), octanol (666 μL) and ammonium hydroxide (150 μL, 28-30% aq.) were mixed together in a 20 mL sample vial with a septum. After thorough mixing, a solution of tetraethylorthosilicate (25 μL) in methanol (500 μL) were added and the reaction mixture was stirred at room temperature for 2 hours. After this time, the reaction mixture was centrifuged for 4 minutes at 14000 rpm and the supernatant was removed by decantation. The isolated solids were resuspended in 5.75 mL of fresh methanol by sonication in an ultrasonic horn bath (1 min, 20% power). This wash step with methanol via centrifugation and resuspension was repeated a further 2 times and the solids were finally resuspended in 5.75 mL of methanol for storage. The solid content was measured by centrifuging 0.2 mL of the sample (as above) in a pre-weighed microcentrifuge tube and carefully removing the supernatant before allowing the solid pellet to dry and re-weighing.

UV/vis spectra in dilute suspension in methanol were measured using a Cary 5000 UV-vis-IR spectrometer. PL spectra at the same concentration in methanol were measured using a Jobin Yvon Horiba Fluoromax-3. Number average diameter of the nanoparticles was determined by Dynamic Light Scattering using a Malvern Zetasizer S.

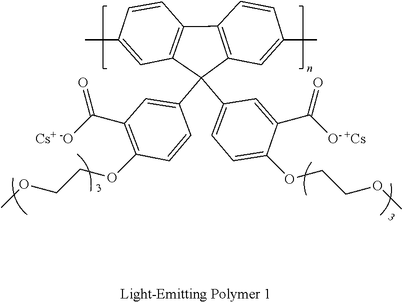

As shown in FIG. 4, the resultant particles had an average diameter of 127 nm as measured by dynamic light scattering.

### Particle Example 2

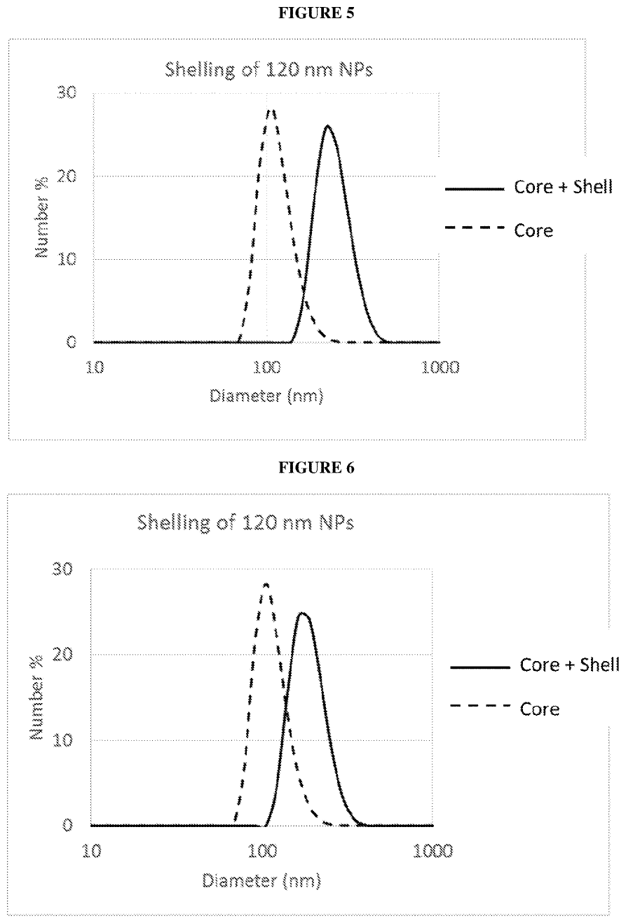

Particles were formed as described in Particle Formation Example 1 except that 120 nm silica was used in place of 60 nm silica (200 μL, 10 mg/mL in water, purchased from Nanocomposix). As shown in FIG. 5, the resultant particles had an average diameter of 246 nm as measured by dynamic light scattering.

Comparative Particle 1

Comparative particles were formed by adsorbing Light-Emitting Polymer 1 onto the surface of the 120 nm silica particles described in Particle Example 2 followed by formation of a silica shell.

120 nm silica nanoparticles (100 μL, 10 mg/mL in water, purchased from Nanocomposix) were rapidly mixed with a solution of Light-Emitting Polymer 1 in methanol (900 μL, 1 mg/mL). The suspension was then centrifuged at 14000 rpm for 3 minutes and the supernatant (containing excess LEP not absorbed to the nanoparticles) was removed by decantation. The solid nanoparticles were resuspended in 1 mL of fresh methanol by sonication in an ultrasonic horn bath (1 min, 20% power).

As shown in FIG. 6, the final particle had a diameter of 189 nm.

The successful incorporation of Light-Emitting Polymer 1 throughout the shells of Particle Examples 1 and 2 was confirmed by measurement of their UV/vis absorption and photoluminescence spectra.

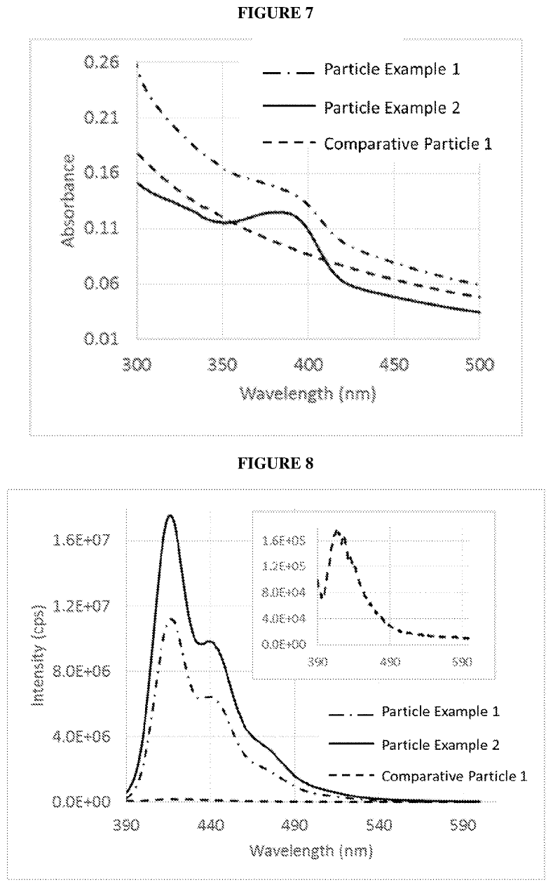

As shown in FIG. 7 a characteristic absorption band of Light-Emitting Polymer 1 can clearly be observed for both Particle Examples 1 and 2 over the scattering signal from the nanoparticles. By contrast, the absorption band from the light-emitting polymer cannot be observed in the comparative nanoparticles in which the light-emitting polymer is only located at the interface between the core and the shell.

As shown in FIG. 8, the brightnesses of Particle Examples 1 and 2 are much higher than that of Comparative Particle 1. The integrated intensity of Comparative Particle 1 (inset in FIG. 8) is 43 times lower than that of Particle Example 2, which has the same 120 nm silica core as Comparative Particle 1.

1. A light-emitting particle comprising a core and a composite shell layer in contact with and surrounding the core wherein the composite shell layer comprises silica and a light-emitting polymer distributed across the thickness of the composite shell layer.

2. A light-emitting particle according to claim 1 wherein the core has a single core layer.

3. A light-emitting particle according to claim 1 wherein the core comprises silica.

4. A light-emitting particle according to claim 1 wherein the particle is a nanoparticle having a diameter of less than 1000 nm.

5. A light-emitting particle according to claim 1 wherein the core and the composite shell layer form a nucleus of the light-emitting particle and wherein the composite shell layer is the outermost layer of the light-emitting particle nucleus.

6. A light-emitting particle according to claim 1 wherein at least one surface group is bound to the composite shell layer.

7. A light-emitting particle according to claim 1 wherein the light-emitting particle comprises a biomolecule binding group bound to the composite shell layer.

8. A powder comprising light-emitting particles according to claim 1.

9. A dispersion comprising light-emitting particles according to claim 1 dispersed in a liquid.

10. A method of forming a light-emitting particle comprising a core and a composite shell layer in contact with and surrounding the core wherein the composite shell layer comprises a light-emitting polymer and silica and wherein the composite shell layer is formed by polymerising a monomer for forming the silica in the presence of the core and the light-emitting polymer in dissolved form.

11. A method of marking a biomolecule, the method comprising the step of binding the biomolecule to a light-emitting marker particle according to claim 1.

12. An assay method for a target analyte comprising contacting a sample with light-emitting particles according to claim 1 and determining any binding of the target analyte to the light-emitting particles.

13. An assay method according to claim 12 wherein the sample contacted with the light-emitting particles is analysed by flow cytometry.

14. An assay method according to claim 13 wherein an amount of target analyte bound to the light-emitting particles is determined.

15. An assay method according to claim 14 wherein the sample comprises a mixture of cells and one or more different types of target cells bound to the light-emitting are identified and/or quantified.

16. The method according to claim 11 wherein the target analyte bound to the light-emitting particles is separated from the target analyte which is not bound to the light-emitting particles.

17. A method of sequencing nucleic acids comprising:
contacting a primed template nucleic acid molecule with a polymerase and a test nucleotide;
incorporating the test nucleotide into a primed strand of the primed template only if it comprises a base complementary to the next base of the template strand;
irradiating the primed strand; and
determining from luminance of the primed strand if the test nucleotide has been incorporated into the primed strand,
wherein the test nucleotide of the irradiated primed strand is bound to a light-emitting particle according to claim 1.
